(19) United States
(12) Patent Application Publication
(10) Pub. No.: US 2014/0314824 A1
(43) Pub. Date: Oct. 23, 2014

(54) POROUS SCAFFOLD FOR TISSUE ENGINEERING AND PREPARATION METHOD THEREOF

(71) Applicants: Se-Kwon Kim, Busan (KR); Venkatesan Jayachandran, Busan (KR)

(72) Inventors: Se-Kwon Kim, Busan (KR); Venkatesan Jayachandran, Busan (KR)

(21) Appl. No.: 13/697,910

(22) PCT Filed: Aug. 30, 2012
(86) PCT No.: PCT/KR12/06924

§ 371 Date: Nov. 14, 2012

(30) Foreign Application Priority Data
Jan. 3, 2012 (KR) 10-2012-0000495

(51) Int. Cl. A61L 27/56; A61L 27/58; A61L 27/20; A61L 27/12

(52) U.S. Cl. CPC A61L 27/56; A61L 27/12; A61L 27/58; A61L 27/20
USPC 424/426; 435/395; 424/602

(57) ABSTRACT

A porous artificial transplant material to replace autogenous bone with excellent biocompatibility, cytocompatibility and biodegradability is provided. More specifically, a porous scaffold for tissue engineering including chitosan/hydroxyapatite-amylopectin (Chitosan/HAp-AP) and a preparation method thereof are provided. The porous scaffold for tissue engineering has cross linkage among chitosan, hydroxyapatite and amylopectin, which provides advantageous effect including superior cell proliferation and transmission, and excellent thermal stability and mechanical strength. Further, considering excellent biocompatibility and biodegradability which do not harm human body, the porous scaffold can be widely used as an artificial transplant material to replace autogenous bone in biomedical field.

### CROSS-REFERENCES TO RELATED APPLICATION

This patent application is a U.S. national phase under 35 U.S.C 371 of PCT/KR2012/006924 filed on Aug. 30, 2012, which claims the benefit of priority from Korean Patent Application No. 10-2012-0000495, filed on Jan. 3, 2012, in the Korean Intellectual Property Office, the disclosure of which is incorporated herein by reference in its entirety.

### BACKGROUND OF THE INVENTION

1. Field of the Invention

The present invention relates to a porous scaffold for tissue engineering which may be used as a porous artificial transplant as a replacement for autogenous bone with superior biocompatibility, cell-compatibility, and biodegradability, and a preparation method thereof.

2. Description of the Related Art

Tissue engineering is a multidisciplinary field that applies basic concepts and techniques of life science and engineering as an approach to understanding of correlation between structures and functions of living tissue, which involves fabrication of a replacement for living tissues for transplantation in vivo with an aim to maintain, improve or restore the human body functions. The tissue engineering is generally understood as a process of taking human or animal tissues, isolating cells from the tissues, culturing the isolated cells in a supporting material (i.e., scaffold) to fabricate cell-scaffold complex, and transplanting the fabricated cell-scaffold complex into human or animal subject.

Tissue engineering is applied to fabricate almost every human organ including artificial skin, artificial bone, artificial cartilage, artificial cornea, artificial blood vessels, and artificial muscles. To ensure that optimum tissues and organs are reproduced, it is important that a scaffold is basically similar to living tissues. An ideal scaffold thus must have, mainly, nontoxicity, mechanical property and porosity.

The ‘nontoxicity’ refers to absence of blood clotting or inflammation in response to implantation of cell-scaffold complex into living tissues, ‘mechanical property’ refers to a strength that can support the cell growth, and the ‘porosity’ refers to a specific structure of a scaffold which is necessary to provide efficient cell adhesion to the scaffold, sufficient space between cells and subsequently, good diffusion of body fluid and oxygen or nutrient supply, and efficient angiogenesis, and subsequently, successful cell growth and differentiation.

Cells are basically cultured two-dimensionally, but in order to culture the cells to the form of tissues or organ, three-dimensional scaffold is necessary. The scaffold has to be formed with many pores to allow cells to adhere inside and outside, and also has to be open to allow supply of nutrients for the cell growth and discharge of waste. In other words, a porous three-dimensional scaffold is necessary.

An ideal scaffold that meets the above-mentioned basic requisites has been researched, among the scaffolds which are similar to the extracellular substrates of animals and which have similar porosity as that of the extracellular substrate. Representative method includes particulate leaching, emulsion freeze-drying, high pressure gas expansion, phase separation and electrospinning.

Meanwhile, a scaffold is required to have biocompatibility and have such a structure that enables cells to adhere and proliferate thereon to form a structure equivalent to tissues or organ. Accordingly, the scaffold can be considered as a substrate for cell growth either ex vivo or in vivo.

Biodegradable polymer is generally used to fabricate the scaffold with the above-mentioned characteristics. Among the biodegradable polymer that meet the above-mentioned physical requirements, widely-used polymers are: collagen, gelatin, hyaluronic acid, chitosan, laminin, keratin, alginate, fibronectin, poly glycolic acid (PGA), polylactic acid (PLA), poly(lactic-co-glycolic acid) (PLGA), poly-ε-caprolactone (PCL), polyamino acids, polyanhydride, polyorthoester, synthetic material of polyurethane, fibrin and fibrinogen, heparin sulfate, chondroitin sulfate, and polysaccharide.

Meanwhile, when implanted in the scaffold of porous structure made from biodegradable polymer, tissue cells form a series of tissues through continuous growth. Accordingly, general reproduction of living tissues mainly includes fabricating a scaffold in the form of living tissues, and implanting tissue cells in the scaffold so that the implanted tissue cells grow therein.

An example can be found in prior art discloses a preparation method of porous scaffold for tissue engineering, and a preparation method of porous scaffold for culturing and delivering cells.

However, the prior art as the ones exemplified above have shortcomings. That is, cell tissues grow rather irregularly, and even if the scaffold has a very thin thickness, it is difficult that the tissue cells grow to the center. Accordingly, a porous scaffold, which can be advantageously used in the tissue engineering or culturing, is still necessary.

### SUMMARY OF THE INVENTION

### Technical Problems

Accordingly, the inventors of the present invention were able to confirm that a scaffold made from chitosan/hydroxyapatite-amylopectin according to freeze-dry method exhibits superior properties including biocompatibility, biodegradability, thermal stability, and high strength, and thus completed the present invention.

Accordingly, an object of the present invention is to provide a porous scaffold for tissue engineering, which comprises chitosan/hydroxyapatite-amyiopectin.

Another object of the present invention is to provide a porous scaffold for tissue engineering, using freeze-dry method.

### Means to Solve the Problems

In order to achieve the objects explained above, the present invention provides a porous scaffold for tissue engineering, comprising chitosan, hydroxyapatite and amylopectin joined with each other in a three dimensional manner.

In one embodiment of the present invention, the chitosan may have molecular weight of 200˜350 KDa and deacetylation of 85˜95%.

In one embodiment of the present invention, the hydroxyapatite may be isolated from bones of Thunnus obesus.

In one embodiment of the present invention, the scaffold may have a porosity of 85˜95%.

In one embodiment of the present invention, the scaffold may have a pore size of 60˜500 μm.

In one embodiment of the present invention, 50˜100 wt. % of hydroxyapatite and 50˜100 of amylopectin may be added per 100 wt. % of chitosan.

In one embodiment of the present invention, a preparation method of a porous scaffold for tissue engineering is provided, which may include steps of: a) dissolving chitosan in acetic acid solution, b) adding hydroxyapatite in the chitosan solution, c) adding amylopectin in the solution formed at step b), and d) preparing the scaffold by freeze-drying the solution formed at step c).

In one embodiment of the present invention, the freeze-drying may be conducted for 24˜48 hours.

In one embodiment of the present invention, after step a), a step of treating the chitosan solution with ultrasonic treatment for 0.5˜2 hours may be additionally included.

In one embodiment of the present invention, after step d), a step of submerging the scaffold formed from the freeze-drying in a basic solution to adjust the scaffold to neutral pH may be additionally included.

### Effect of the Invention

The porous scaffold for tissue engineering according to the present invention provides advantageous effects in that the scaffold has conjugate porous structure via bridge bonding among chitosan, hydroxyapatite and amylopecting, which in turn results in efficient cell proliferation and transmission, and the scaffold also has superior thermal stability and mechanical strength. Further, the scaffold according to the present invention can be widely used as an artificial transplant for replacement of autogenous bone in biomedical field considering its superior biocompatibility and biodegradability which do not harm human body.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 illustrates porosity of chitosan, chitosan/HAp and chitosan/HAp-AP scaffold according to an embodiment of the present invention;

FIG. 2 illustrates water absorbability and retainability of chitosan, chitosan/HAp and chitosan/HAp-AP scaffold according to an embodiment of the present invention;

FIG. 3 illustrates TGA result regarding thermal stability of chitosan, hydroxyapatite, amnylopectin and conjugate scaffolds thereof according to an embodiment of the present invention;

FIG. 4 presents FTIR spectra of chitosan, hydroxyapatite, amnylopectin and conjugate scaffolds thereof according to an embodiment of the present invention;

FIG. 5 presents XRD spectra of chitosan, hydroxyapatite, amnylopectin and conjugate scaffolds thereof according to an embodiment of the present invention;

FIG. 6(I) presents optical microscopic images of scaffolds, FIG. 6(II) presents optical microscopic images of MTT-analyzed cells on scaffolds and FIG. 6(III) presents optical microscopic images of Hoechst 33342 stained cells on scaffolds according to an embodiment of the present invention;

FIG. 7(I) presents SEM images of chitosan, hydroxyapatite, amnylopectin, FIG. 7(II) presents SEM images of respectively-prepared scaffolds and FIG. 7(III) presents SEM images of MG-63 cell line grown on scaffolds, according to an embodiment of the present invention;

FIG. 8 presents result of measuring differentiation of MG-63 cell line on scaffold by MTT assay according to an embodiment of the present invention;

FIG. 9 presents expression level of alkaline phosphatase and type-I collagen on scaffolds according to an embodiment of the present invention.

### DETAILED DESCRIPTION OF THE INVENTION

The present invention relates to a porous artificial transplant material as a replacement for autogenous bond, and more specifically, to a porous scaffold for tissue engineering comprising chitosanhydroxyapatite-amylopectin (chitosan/Hap-AP) and a preparation method thereof.

The “tissue engineering” as used herein generally refers to a process of fabricating equivalent material to tissues or organs by seeding cells on or in a scaffold suitable for transplantation. Generally, most tissues are made of several hierarchical cells, and when isolated, most cells can return to original shape in small scale. However, a supporting framework (i.e., “scaffold”) is necessary for the cells to shape larger forms. The supporting framework has to collect and adhere the cells both in in vivo and ex vivo, be a potential place for the cells to develop into tissues, and enable adhered cells and the adjacent host cells to form new tissues.

The “scaffold” as used herein refers to a physical support and adhesion substrate prepared to enable culturing and implantation of tissue cells ex vivo and in vivo. The scaffold is generally used in cell implantation for the restoration of human tissues. The importance of the scaffold is very high particularly in terms of mass culture and proliferation of the cells. This is because the use of scaffold is the only available way to meet the requirement that as many as possible cells be obtained ex vivo and allowed to differentiate to new autologous tissues, to achieve more effective utilization of the stem cells or cells collected by biopsy in patients.

In order to be used in the reproduction of human tissues include, a scaffold material has to play a role of a substrate or framework to allow tissue cells to adhere on the surface thereof and form tissues with three-dimensional structure, and also play a role of a wall between the implanted cells and the host cells, which means that the scaffold has to be nontoxic and biocompatible so as not to incur blood clotting or inflammatory response when implanted. Additionally, the scaffold also has to have biodegradability. That is, the scaffold has to decompose completely in vivo at desired time, upon completing intended functions and roles as tissues.

The ideal properties of biocompatible scaffold include abilities to: support growth of cells ex vivo or in vivo; support growth of a wide range of cell types of systems; attain various levels of flexibility or rigidity as desired; degrade at different various levels; enter into targeting area in vivo without causing secondary damage; and provide a storage or carrier to deliver cells and/or bioactive substance. Further, a scaffold for bone tissue engineering is required to have a high porosity and interconnected cancellous structure to construct a biological environment which allow cell adhesion, proliferation, tissue growth and appropriate nutrient supply.

The biodegradable polymer best meets the above-mentioned requirements. The biodegradable polymer has an ability to reproduce tissues to a determined size or form, and also provides temporary place for cell adhesion and expansion either in vivo or ex vivo. Accordingly, the biodegradable polymer is a good material to be used as a synthetic extracellular substrate for cell proliferation and cell differentiation.

According to the present invention, a novel scaffold having all the properties explained above that are required for an extracellular matrix of bone is prepared, which is a chitosan/hydroxyapatite (HAp)-amylopectin (AP) comprising chitosan, hydroxyapatite (HAp), and amylopectin (AP) using freeze dry method, and without applying complicated processing.

Accordingly, the present invention provides a porous scaffold for tissue engineering comprising chitosan, hydroxyapatite and amylopectin joined with each other in three dimensional manner.

In one embodiment of the present invention, the materials used in the preparation of a scaffold act similarly to all the properties of the bone extracellular substrate including biocompatibility, biodegradability and bioactivity. These properties play a crucial role in terms of performance of the transplanted bone tissues, and the polymer-based scaffold with biocompatibility and biodegradability maintains mechanical property and entirely absorbed into a body upon completing a task.

In the chitosan/HAp0AP conjugate scaffold according to the present invention, because the constituent components of the scaffold, i.e., chitosan, HAp and AP chemically react with each other to form three-dimensionally conjugated porous structure, the scaffold is designed sturdier and more stable enough to endure mechanical stress, and has reinforced strength. Further, the porosity of the scaffold according to the present invention provides surface suitable for the cell adhesion and growth.

Further, it is possible to adjust biodegradability of the chitosan/HAp-AP conjugate scaffold according to the present invention by adding HAp and AP to chitosan polymer-based scaffold.

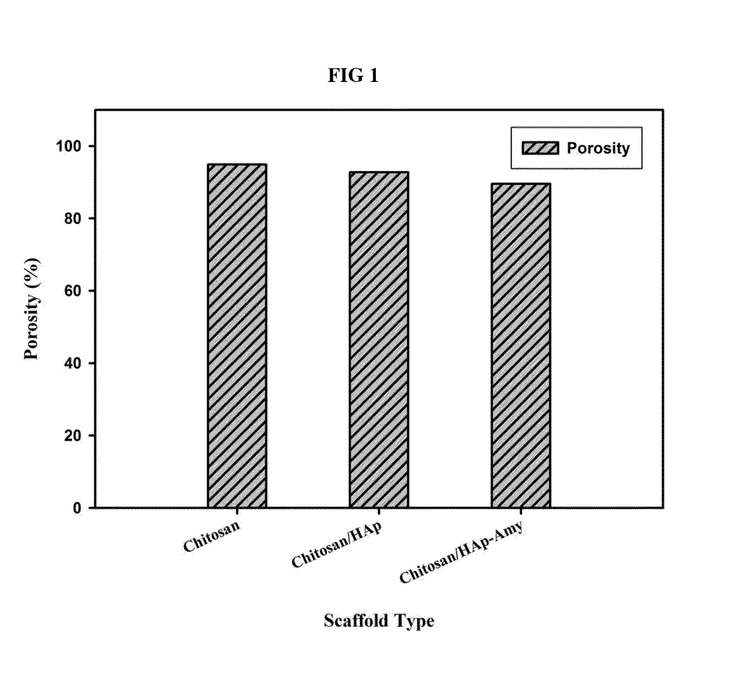

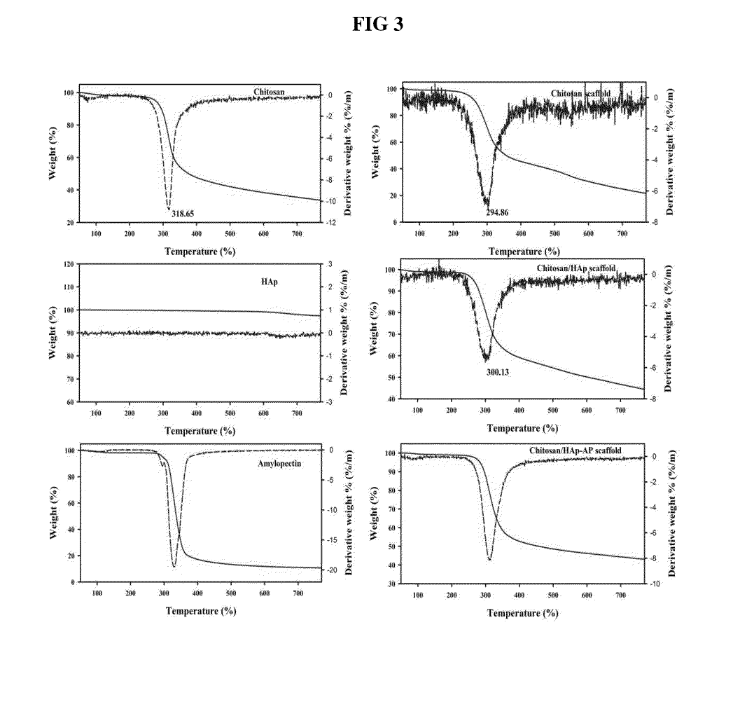

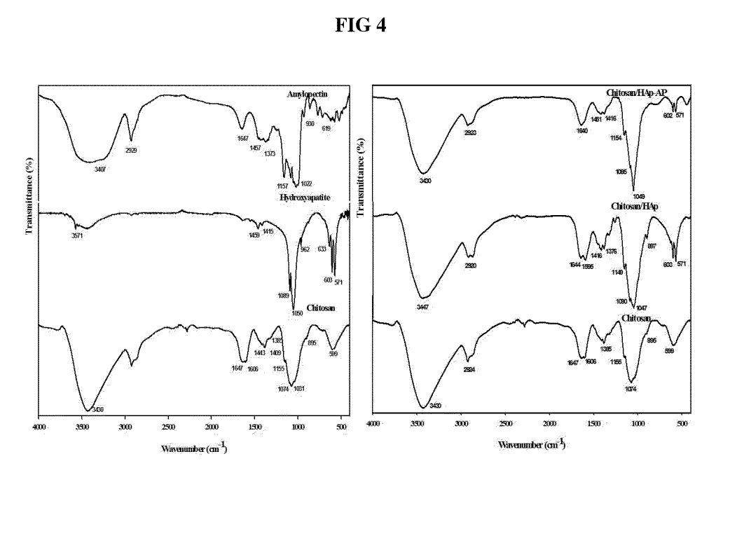

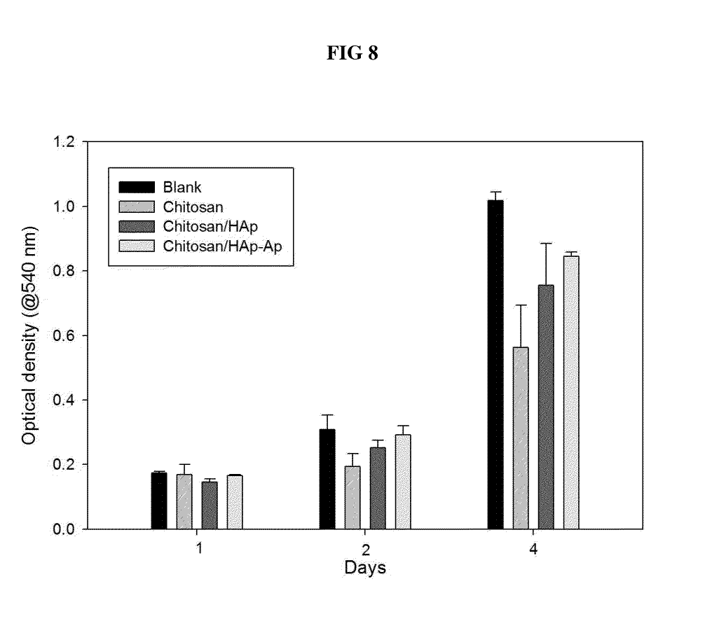

In one embodiment of the present invention, as a result of investigating major properties of a chitosan/HAp-AP conjugate scaffold prepared with freeze dry method, the liquid displacement method revealed that the scaffold has 89% or higher porosity (FIG. 1), and thermogravimetric analysis (TGA) revealed that the conjugate scaffold is stable at high temperature (FIG. 3). Further, FT-IR spectrometry revealed that chitosan/HAp-AP scaffold has cross linking among chitosan, HAp and AP (FIG. 4). In comparing the thermal stability, porosity and cell proliferation of the scaffold, ex vivo cytotoxicity assay revealed that MG-63 cell line exhibited no cytotoxicity (FIG. 8). Considering the above-mentioned findings, the chitosan/HAp-AP scaffold according to the present invention is considered potentially very important particularly in the bond tissue engineering field, since this provides good thermal stability, interconnected cancellous structure, adjustable biodegradability, and reinforced cell proliferation.

In one embodiment of the present invention, chitosan may have intermediate molecular weight, and preferably 200˜350 KDa of molecular weight. In a specific embodiment of the present invention, chitosan with 310 KDa of molecular weight is used. Further, deacetylation in a range of 85˜95% may be used, and in one embodiment of the present invention, the deacetylation of 90% is used.

Further, hydroxyapatite may be separated from bone or artificially synthesized, and in one embodiment of the present invention, isolated from the bone of Thunnus obesus.

In one embodiment of the present invention, chitosan and hydroxyapatite, and amylopectin may be mixed in a ratio of 50˜100 wt. % of hydroxyapatite, and 50-100 wt. % of amylopectin per 100 wt. % of chitosan.

In one embodiment, the porous chitosan/HAp-AP scaffold has network-shaped pores, with the pore size increasing in accordance with addition of AP. The size of the pores may preferably be 60˜500 μm, and the structure and size of the pores as explained above advantageously contribute to the cell growth and nutrient supply so that the cells can growth into the scaffold efficiently. That is, it is very important in the tissue reproduction via maintenance of tissue volume, cell proliferation, and gene and protein transmission.

The porosity of a scaffold decreases in accordance with addition of hydroxyapatite and amylopectin, and preferably be 85-95% to achieve the desired function as a transplant material. If porosity is below 85%, pore size is small and irregular, which does not make up interconnected pore network structure, while if porosity exceeds 95%, the scaffold has too many pores to provide appropriate strength. Accordingly, the porosity is preferably at 90%.

Meanwhile, a preparation method of porous scaffold for tissue engineering according to the present invention includes steps of: a) dissolving chitosan in acetic acid solution; b) adding hydroxyapatite in the chitosan solution; c) adding amylopectin in the solution prepared at step b); and d) preparing a scaffold by freeze-drying the solution prepared at step c).

First, chitosan is dissolved in acetic acid, along which ultrasonic treatment may additionally be performed to remove air bubbles which may occur in the process of dissolving by stirring. The ultrasonic treatment may be performed for 0.5˜2 h. In one embodiment of the present invention, the ultrasonic treatment was performed for 30 min.

Hydroxyapatite is added to the chitosan solution and then amylopectin is added.

Next, the prepared solution is freeze-dried to form a scaffold, in which the freeze-drying is begun at about −80° C., performed about 24˜48 h, in a freeze dryer. In one embodiment of the present invention, the freeze drying was performed at −80° C., 48 h.

The scaffold obtained as a result of freeze drying is submerged in basic solution to adjust pH to neutral, and after washing and drying, the chitosan/HAp-AP scaffold according to the present invention is prepared.

The method according to the present invention may additionally include a step of sterilizing the scaffold at an appropriate time point using an appropriate method.

Further, the scaffold according to the present invention may be cut and shaped to a desired size and shape.

During initial cell adhesion process, the chitosan/HAp-AP scaffold according to the present invention provides superior cell proliferation and transmission in the conjugate scaffold, compared to other chitosan scaffolds, due to interconnected porous structure thereof. Further, the scaffold according to the present invention can be widely used as an artificial transplant for autogenous bone replacement in the biomedicine field, considering good thermal stability and improved mechanical strength thereof, and also considering that superior biocompatibility and biodegradability thereof lead to no harm to human body.

Hereinbelow, the present invention will be explained in detail with reference to examples and drawings. However, the examples are given only for the purpose of explaining the present invention, and should not be construed as limiting the invention.

### Example 1

### Preparation of a Scaffold According to the Present Invention

<1-1> Materials

In the present invention, chitosan, neutral HAp and AP were used to prepare a scaffold. Chitosan with middle molecular weight (˜310 KDa, deacetylation 90%) was purchased from Kitto life Co., Ltd (South Korea). HAp was isolated from the bones of Thunnus obesus by thermal sintering, and amylopectin was purchased from Sigma Chemicals.

Human osteocyte-like cell line (MG-63) was provided by ATCC (Manassas, Va., USA), and DMEM (Dulbecco's Modified Eagle's Medium) was provided by Gibco BRL, Life Technology. MTT (3-(4,5-dimethyl-2-yl)-2,5-diphenyltetrazolium bromide) was purchased from Molecular Probes (Eugene, Oreg., USA), and Bisbenzimide Hoechst 33342 stain was purchased from SigmaAldrich (St. Louis, Mo., USA). Glutaraldehyde 25% was purchased from Junsei Chemical (Japan). Further, all the reagents used in the experiment were analytical grade.

<1-2> Preparation of Chitosan Scaffold

First, 2.5 g of chitosan with middle molecular weight was dissolved in 2% acetic acid solution (250 ml) and stirred overnight using a stirrer (RW 20.n Labortechik). After the stirring, ultrasonic treatment was performed 1 h. The solution was transported to small petri dish (35×10 mm). After freezing at −80, 48 h, freeze drying was performed to form a scaffold.

The scaffold was submerged in 10% sodium hydroxide, washed with generous amount of water to neutral pH, and freeze dried against in preparation for the next experiment.

<1-3> Preparation of Chitosan/HAp Scaffold

In order to prepare chitosan/HAp scaffold, 2.5 g of chitosan with middle molecular weight was dissolved in 250 ml of acetic acid solution, stirred overnight, and ultrasonic treated for 1 h to remove air bubbles. 2.5 g of HAp isolated from the bones of Thunnus obesus was dispersed in 50 ml of water, and transported in chitosan solution with care by a dropper. The following process was same as the process of preparing chitosan scaffold.

<1-4> Preparation of Chitosan/BAp-AP Scaffold

In order to prepare chitosan/HAp-AP scaffold, 2.5 g of AP was slowly added to chitosan/HAp solution and the mixed solution was stirred overnight. The following process was same as the process of preparing chitosan scaffold.

### Example 2

### Gross Examination of Scaffold

Chitosan and conjugates scaffolds thereof prepared using freeze drying method were observed as stiff and elastic from a gross examination.

Further, when all the scaffolds are submerged in 0.1M PBS, chitosan scaffold was more supple and swelled faster compared to chitosan/HAp and chitosan HAp-AP scaffolds. This is because addition of HAp and AP keeps chitosan tissue firmer.

Chitosan and chitosan/HAp scaffold were colorless, while chitosan/HAp-AP scaffold appeared white due to dispersion of AP in the polymer and ceramic matrix.

### Example 3

### Measurement of Porosity

Porosity is the most important parameter in a bone tissue engineered scaffold. Porous scaffold material plays a very important role in the tissue engineering due to maintenance of tissue volume, provision of temporary mechanical function, cell adhesion and proliferation and transmission of genes and proteins. The total porosity of each of the scaffolds prepared at <Example 1> was measured using liquid displacement method.

First, volume of ethanol and dry weight of the scaffold were measured. Next, after keeping the scaffold in dehydrated alcohol for 48 h until the scaffold absorbed alcohol and saturated, the weight of the scaffold was measured again. Lastly, the porosity of the sample was calculated by:

Porosity=(V₁−V₃)/V₂−V₃

where, V₁ denotes initially-measured weight of scaffold,

V₂ is a sum of weights of ethanol and scaffold submerged therein, and

V₃ is a weight of ethanol removed of scaffold.

Three samples were analyzed regarding all the scaffolds, and average porosity of the respective samples was obtained.

As a result, the porosity of chitosan scaffold was 94.87%, while the porosity of chitosan/HAp scaffold and chitosan/HAp-AP scaffold were 92.87% was 89.54% which were lower than that of chitosan (see FIG. 1 and Table 1).

The total porosities of chitosan/HAp scaffold and chitosan/HAp-AP scaffold were lower than that of chitosan due to chemical interaction between scaffold materials. The chemical interaction occurs between NH₂ group of chitosan and —OH group of HAp, which decreases the porosity of a conjugate scaffold. Further, the reason for further decrease of the porosity of chitosan/HAp-AP scaffold than that of chitosan/HAp scaffold is because of the branched polysaccharide unit present in AP, and this is considered to be a result of regulative chemical interaction among molecules.

TABLE 1

| | | | Thermal | | |
|---|---|---|---|---|---|
| | | Total | degradation | Biodegrad- | Pore |
| | | porosity | point | ability | size |
| No. | Scaffold | (%) | (° C.) | (%) | (μm) |
| 1 | chitosan | 94.87 | 294.86 | 12 | 40~400 |
| 2 | chitosan/HAp | 92.78 | 300.13 | 4 | 60~180 |
| 3 | chitosan/HAp- | 89.54 | 312.49 | 6 | 80~500 |
| | AP | | | | |

### Example 4

### Water Absorbability and Retainability

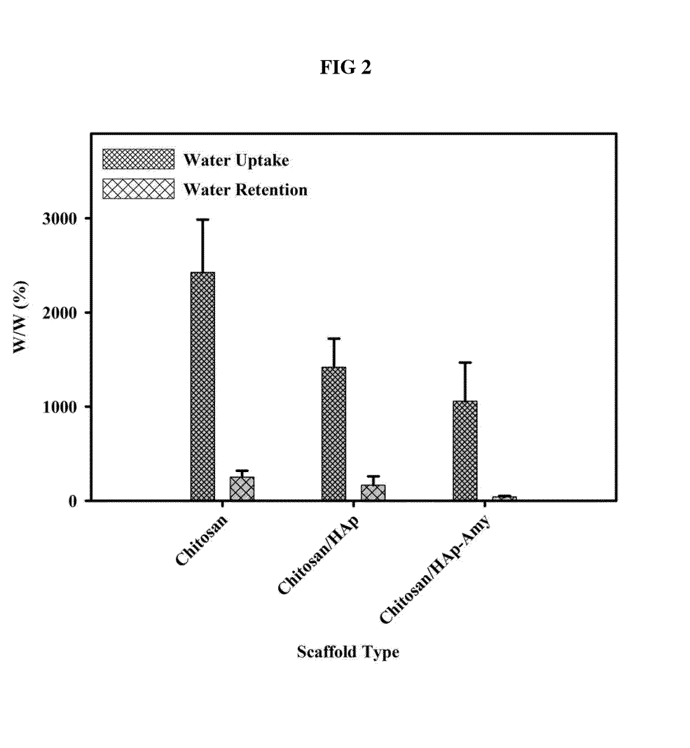

The ability of a scaffold to absorb and retain water is essentially required for not only absorption of bioactive liquids, but also transmission of nutrients and metabolites through the scaffold. The water absorbability and retainability of a scaffold were thus examined as explained below and the result is presented in FIG. 2.

First, the weight (W_{dry}) of a dried scaffold was measured, and the scaffold was submerged in distilled water for 24 h. Next, the scaffold was gently removed from the beaker, and placed on a wire mesh rack. Excess water was drained and after 5 m, the weight (W_{wet}) of the scaffold was measured to confirm water absorption. The wet scaffold was moved to the centrifuge tube including filter paper on a bottom to measure water retainability. After centrifugation at 500 rpm, 3 m (Combi 514-Hanil Science industrial), the weight (W′_{wet}) was directly measured.

In an equilibrium, the water absorbability (EA) and retainability (ER) ratios were calculated by:

EA=[W_{wet}−W_{dry}/W_{dry}]×100

ER=[W′_{wet}−W_{dry}/W_{dry}]×100

As a result, chitosan scaffold had higher water absorbability and retainability than chitosan/HAp scaffold and chitosan/HAp-AP scaffold. From the above finding, it was confirmed that water absorbability was decreased in accordance with the addition of HAp and AP to pure chitosan. It was also observed that the water absorbability decreased as the HAp content increased.

### Example 5

### In Vitro Biodegradation Test

The biodegradation of a scaffold was measured using lysozyme and 0.1M phosphate buffer solution (hydrogen sodium phosphate (monobasic) and hydrogen sodium phosphate (dibasic) and pH 7.2 PBS).

The scaffold sample was submerged in falcon tube containing 10 ml PBS and lysozyme and vibrated at 37.0±0.5 and kept. After 7, 14 and 21 days of submerging, the sample was taken and washed with deionized water, and after freeze drying, the weight was measured again. Three similar samples were used for all the scaffolds.

The weight loss (WL) was calculated by:

W_{L}=(W₀−W₁)/W₀×100%

where, W₀ refers to sample weight before submerging, and W₁ refers sample weight after submerging.

As a result, it was confirmed that chitosan was decomposed by PBS which is similar to human body fluid and intracellular environment of tissues. At 21 d, the chitosan scaffold was observed to have been decomposed by approximately 12% of original weight, and chitosan/HAp scaffold was decomposed by about 4%, and chitosan/HAp-AP scaffold was decomposed by about 6% (see Table 1).

From the above finding, it was confirmed that the addition of HAp and AP deteriorates biodegradability of a scaffold.

### Example 6

### Analysis of Thermal Stability of Scaffold

Thermal stability of the scaffold was measured by thermogravimetric analysis (TGA) and the result is shown in FIG. 3.

The thermogravimetric and derivative thermogravimetric (TGA-DTG) analysis was conducted using Pyris 1 TGA analyzer (Perkin-Elmer Inc., USA) at constant heating rate of 10° C. min under continuously flowing nitrogen, with scan range of 50˜800° C.

As a result, TGA and dTG curves revealed reduction of weight of chitosan scaffold at 294.86° C. which correspond to moieties of glucosamine.

The reduction of weight of chitosan/HAp scaffold was observed at 300.13° C., and the reduction of weight of chitosan/HAp-AP scaffold was observed at 321.49° C. The difference of point of degradation between chitosan/Hap and chitosan/HAp-AP is attributable to shared interactions among chitosan, HAP and AP. From the above findings, it was confirmed that thermal stability of scaffold is increased by an addition if AP.

Further, at 800° C., chitosan degrades about 80%, while chitosan/Hap and chitosan/HAp-AP degrade about 55% and abou 60% respectively. The degradation of the chitosan/HAp and chitosan/HAp-AP scaffold was limited at high temperature and this is because of the presence of HAp added thereto which is stable to heat. From the above findings, it was confirmed that the conjugate scaffold is very stable at high temperature.

### Example 7

### Analysis of Scaffold by FTIR

Fourier Transform Infrared Spectroscopy (FTIR) was measured to investigate interaction among the molecules constituting a scaffold. The stretching frequency of the scaffold was measured within 400˜4000 cm⁻¹ range with Fourier Transform Infrared Spectroscopy (Perkin Elmer, USA) [symbol] spectrum GX spectrometer and the resultant measurements are shown in FIG. 4.

On the IR spectrum of pure chitosan, characteristic peaks appeared around 3430 cm⁻¹ and 2924 cm⁻¹ which correspond to N—H stretching vibration and OH stretching vibration coupling, and the characteristic peaks of amide I and amide II appeared at 1647 cm⁻¹ and 1606 cm⁻¹, respectively. The sharp peak appearing at 1409 cm⁻indicates CH₃ symmetrical deformation mode, and 1074 cm⁻¹ and 1031 cm⁻¹ indicate C—O stretching vibration v(C—O—C).

IR spectrum of HAp indicated characteristic absorbance band of HAp. Many bands appearing in the HAp spectrum (3571, 1459, 1415, 1089, 1050, 962, 633, 603 and 571 cm⁻¹) appeared similar to the previously-reported HAp reference spectrum.

The IR spectrum of AP had peaks at 800 cm⁻¹, 800˜1500 cm⁻¹ (fingerprint region), 2800˜3000 cm⁻¹ (C—H stretching area), 3000˜3600 cm⁻¹ (O—H stretching area), and showed almost identical band as that of starch.

As a result of conducting FTIR on the scaffold, chitosan/HAp scaffold showed mixed characteristic peaks of chitosan and HAp, which clearly indicates that HAp particles are highly and evenly dispersed in the matrix.

The characteristic peaks were observed at 571 cm⁻¹ and 603 cm⁻¹, and small shifting was observed at not only 1047 cm⁻¹, 1090 cm⁻¹ and 1416 cm⁻¹ (corresponding to HAp), but also 2920 cm⁻¹, 1644 cm⁻¹, 1595 cm⁻¹, 1145 cm⁻¹ (corresponding to stretching frequency of chitosan). In chitosan/HAp, the stretching frequency of chitosan decreases, which indicates that intermolecular and co-ordination chemical interactions are possible between chitosan and calcium ions of HAp particles in the conjugate scaffold. This chemical interactions make the conjugate scaffold stronger and more stable, and chitosan in the conjugate scaffold not only plays a role of a back bone, but also maintains the strength of the scaffold.

Meanwhile, the peaks of chitosan/HAp-AP scaffold observed at 571 cm⁻¹, 602 cm⁻¹, 1049 cm⁻¹ and 1085 cm⁻¹ derive from HAp crystals, and the decrease of chemical stretching frequency observed at 1146 cm⁻¹ is due to several chemical interactions with moieties of chitosan. Further, in chitosan/HAp-AP scaffold, maximum transmissivity of HAp crystals is observed, which is due to high dispersion of ceramic matrix in polysaccharide (chitosan, AP). From the above findings, it was confirmed that chitosan and HAp and AP chemical interact with each other.

### Example 8

### X-Ray Diffraction (XRD) Analysis

The phase and crystallinity of the scaffold were measured with X-ray diffractometer (PHILIPS X'Pert-MPD diffractometer, Netherland).

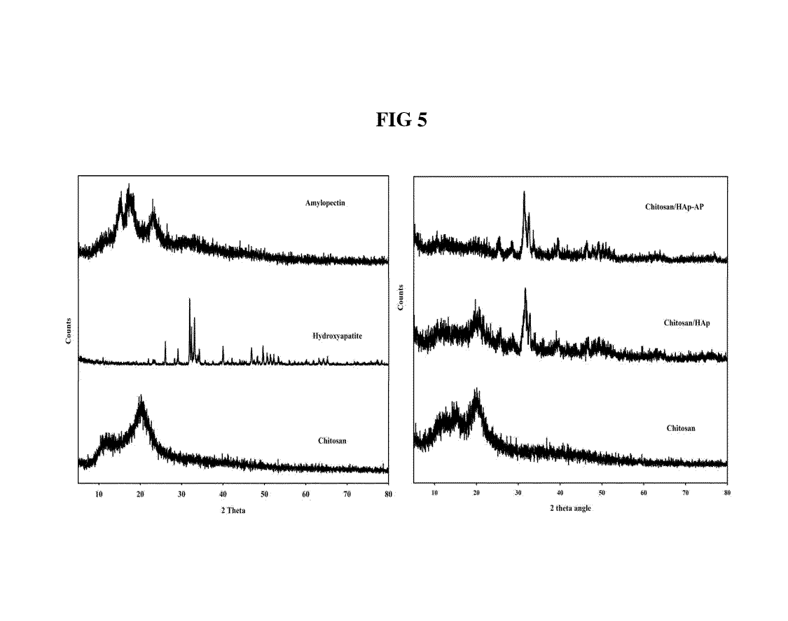

The diffraction patterns of chitosan, neutral HAp, AP, chitosan/HAp scaffold, and chitosan/HAp-AP scaffold are shown in FIG. 5.

As a result, in chitosan, three major peaks appeared at 10.5, 20.0 (maximum intensity) and 22.5. The HAp diffraction pattern was identical to the previously-reported result. Further, due to amorphous property of polysaccharide, AP displayed wide diffraction bands similar to those of chitosan scaffold, in which the diffraction peak of maximum intensity appeared at 17.2, and also at 15.2, 18.3, 23.0 except several other additional peaks. AP diffraction peaks are very wide, thus indicating the high amorphous property and low crystallinity of AP.

Further, the chitosan diffraction peaks of chitosan/HAp scaffold and chitosan/HAp-AP scaffold both appeared at 20.0, and the maximum diffraction peak corresponding to the highly crystalline HAp diffraction pattern appeared at 31.4 in the chitosan/HAp-AP scaffold.

The crystallinity of HAp in the conjugate scaffold appeared relatively lower than that of pure HAp, and this is considered to be attributed to the reduction of crystallinity due to HAp stirring in the process of preparing scaffold.

### Example 9

### Optical Microscope Analysis

Scaffold, MTT treated cells and Hoechst stained cells were examined under optical microscope (CTR 6000; Leica, Wetzlar, Germany) and visualized.

<9-1> Optical Microscope Examination

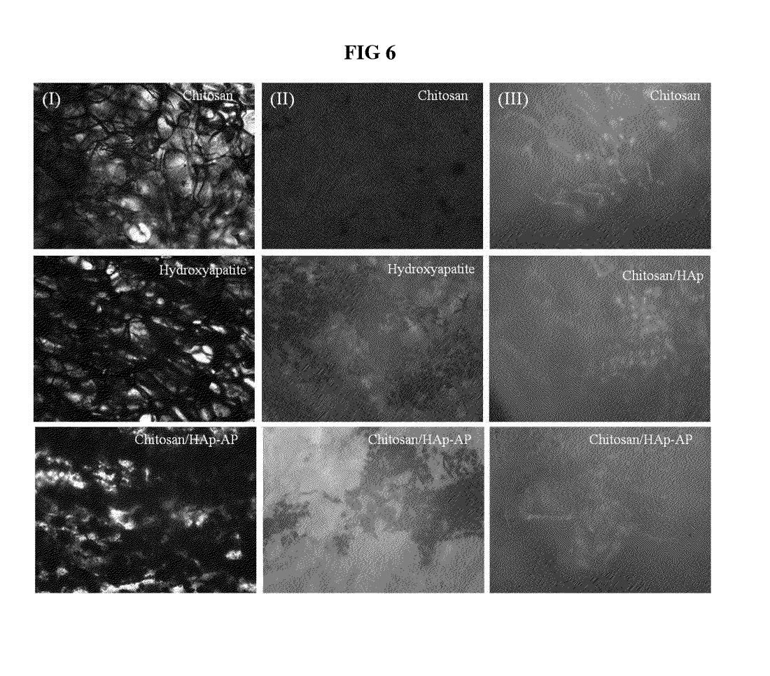

HAp and AP dispersion in the chitosan matrix was observed under optical microscope and the result is shown in FIG. 6(I).

As a result, while the incident ray of light of the optical microscope penetrates easily into chitosan matrix with high porosity, the ray is reflected from chitosan/HAp, chitosan/HAp-AP scaffold. This is because of the presence of HAp. Further, appropriate dispersion and distribution of AP in the polymer and ceramic matrix was observed and this is in agreement with the result of SEM observation.

<9-2> NFT Analysis

To investigate biocompatibility of the materials to be used, cytotoxicity was measured with MTT assay and the result is shown in FIG. 6 (II). The MTT assay is based on mitochondrial reduction in which MTT salt is reduced to formazan crystal in living cells.

MTT assay utilizes mitochondrial ability to reduce yellow, soluble MTT tetrazolium to purple color, insoluble MTT formazan with dehydrogenate enzymatic action. The reduction occurs when the mitochondria reductase is activated. The light absorption of MTT formazan reaches maximum at a wavelength of 540 nm, at which the measured absorbance indicates the concentration of living and actively metabolizing cells.

From the above findings, it was confirmed that the conjugate scaffold had higher amount of formazan crystals than chitosan scaffold, which can be explained by the fact that the cell proliferation is induced in the chitosan/HAp-AP scaffold.

<9-3> Hoechst Staining

Hoechst staining was conducted to examine cell growth in the conjugate scaffold and the result is shown in FIG. 6 (III).

Hoechst stain 33342 (closely related to bis-benzimide), a fluorescent DNA marker dye generally used to visualize nucleus and mitochondria, was used to find living cells on the scaffold.

Three samples of each scaffold were placed in 24 well cell culture plate, and MG-63 cell line (2×10³ cells/well) was seeded drop by drop. Further, the scaffold was incubated 37° C., 4 h to allow the cells to adhere onto the scaffold, and after the incubation, 1.5 ml of new media were added to the respective wells. After 7 days of incubation, media were removed from the wells, the scaffold with the cells thereon was washed with PBS two times, and immobilized in 2.5% glutaraldehyde overnight at −4° C. The cells were stained with 10 μg/ml fluorescent DNA-marker dye, Bisbenzimide Hoechst 33342, and after 20 m of incubation at 37° C., 20, pycnosis/condensation of nucleus appeared. The Hoechst-stained cells were observed visually, and examined under optical microscope (CTR 6000; Leica, Wetzlar, Germany).

As a result, it was clearly observed that the conjugate scaffold (chitosan/HAp, chitosan/HAp-AP) had higher cell concentration than the chitosan scaffold. However, several apoptosis cells were observed in the conjugate scaffold and this is considered to be due to high confluency of the cells.

### Example 10

### Morphological Analysis of Scaffold

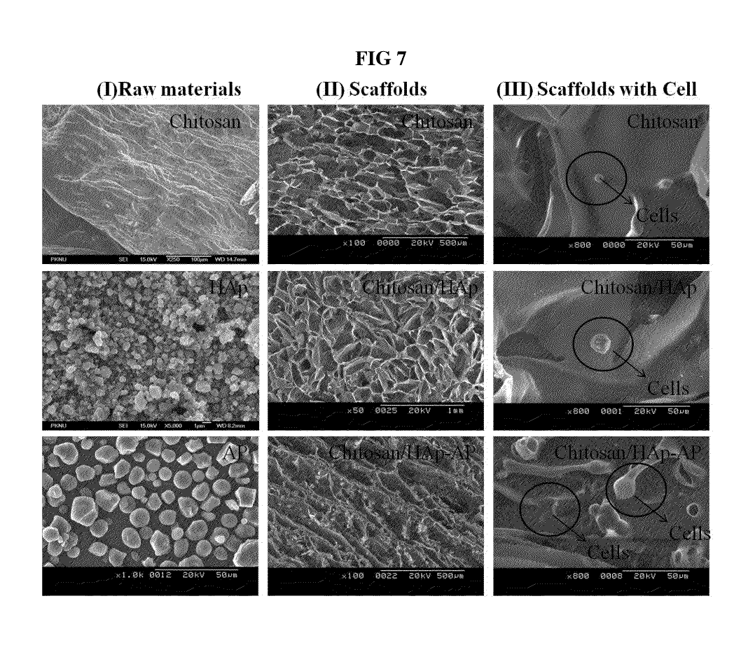

To investigate the morphology and porosity of the scaffold, Scanning Electron Microscopy (SEM) (HITACHI S-2400, Japan) was used and the result of analysis is shown in FIG. 7.

As a result, chitosan was observed to have a structure of high density due to high molecular weight and joined tissues, and combination of micro- and nano-sized materials was observed in HAp and AP (FIG. 7(I)). The micro- and nano-sized crystalline structure is far more advantageous for the effective regulation of a scaffold, than an example consisting of macro-sized materials only.

Further, the SEM images of chitosan, chitosan/HAp, chitosan/HAp-AP scaffold revealed the cell growth on the scaffold thereof (FIG. 7(III)). The conjugate scaffold has almost equivalent porous structure and excellent interconnected three-dimensionalized structure, and HAp particles were dispersed evenly in the chitosan matrix. The interconnected porous structure was observed in all the conjugate scaffolds, and in addition to the even dispersion of HAp particles, AP was also observed to be in even dispersion in the chitosan matrix. This is considered to be attributable to the charge-bearing property of the chitosan network.

Meanwhile, the pore sizes of chitosan/HAp scaffold) chitosan/HAp-AP scaffold varied within respective ranges of 60-180 and 80-500. From the above findings, it was confirmed that when AP is added to polymer and ceramic matrix, the pore size increases compared to chitosan/HAp scaffold. As the pore size increases, it is more advantageous for cell growth and nutrient supply.

### Example 11

### In Vitro Cell Differentiation Analysis

The cell proliferation and biocompatibility of the scaffold were investigated with MTT assay, and the result is shown in FIG. 8.

The osteocyte-like cell line MG-63 was cultured in DMEM containing 10% FBS, 2 mM glutamine and 100/ml penicillin-streptomicin at 37 and under 5% CO₂ wet air condition. The in vitro cell differentiation in the scaffold was investigated with MTT assay. MG-63 cell line and 90% confluency obtained from cell culture flask (75 cm², filter cap) were used to investigate cytocompatibility and cell differentiation of the conjugate scaffold.

Before cell seeding, the scaffold was placed in 24-well culture plate, 400 media were added, and incubated in a wet incubator (5% CO₂ and 95% air) at 37° C., for 4 h.

100 μl of 2×10³ cell was seeded on the scaffold drop by drop so that the cells were distributed across the scaffold. Accordingly, the scaffold with the cells seeded thereon, was incubated in a wet incubator (5% CO₂) at 37° C. for 4 h to allow the cells adhere to the scaffold.

The media were replaced with new ones everyday. The three replicates were used in the experiment of each scaffold type. Further, cell viability on the scaffold was measured with MTT assay. The media on the scaffold were removed everyday, and incubation continued in the fresh media containing 400 μl MTT (1 mgml¹ medium) at 37° C. 4 h in the dark. After removal of nonreactive stains, color-expressing solution was added with 400 μl of DMSO and the purple-colored formazan lysate was decomposed. The absorbance of the solution was measured at 540 nm using spectrophotometer and aGENios microplate reader (Tecan Austria GmbH, Austria).

As a result of investigating with osteocyte-like cell (MG-63) in vitro, it was observed that the scaffold did not exhibit toxicity in the tissue. Further, in the early phase of cell adhesion, the conjugate scaffold had superior cell proliferation and transmission due to interconnected porous structure thereof, compared to chitosan scaffold.

### Example 12

### Production of Alkaline Phosphatase and Typo-I Collagen

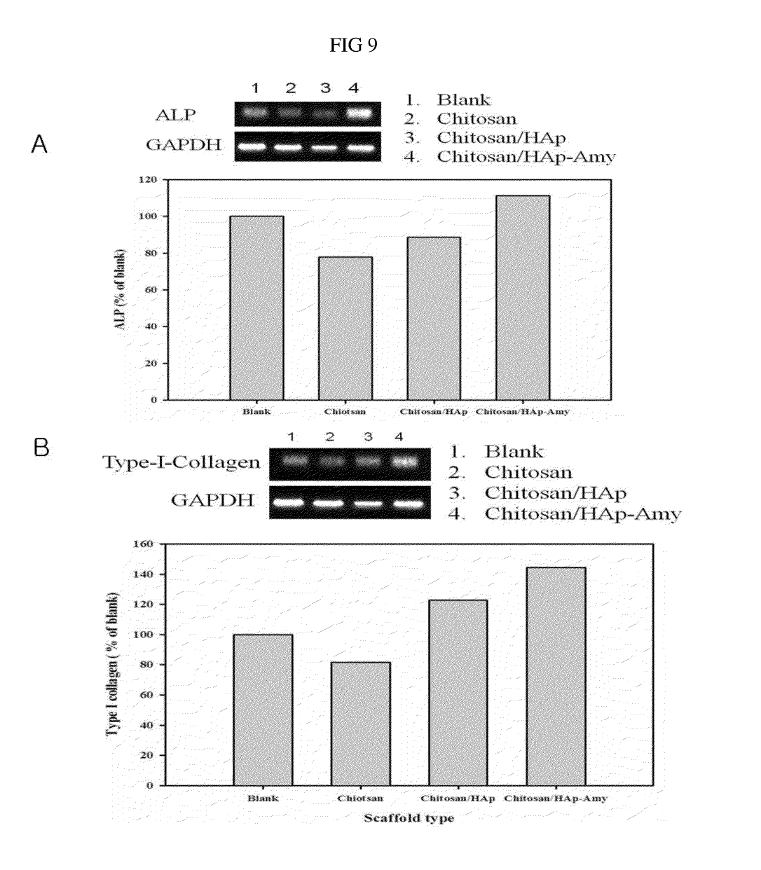

Polymerase chain reaction (PCR) assay was conducted to investigate alkaline phosphatase and type-I collagen productions in the conjugate scaffolds and the result is shown in FIG. 9.

Total cellular RNA was extracted using trizol reagent (Invitrogen Co., CA, and USA). Simply put, total RNA (2 μg) was inverse transferred in a master mixture containing 1×RT buffer, 1 mM dNTPs, 500 ng oligo(dT), 140U M-MLV reserve transcriptase and 40U RNase inhibitor at 42° C. for 60 m, and at 72° C. for 5 m, using automatic Whatman thermo cycler (Biometra, UK).

Target cDNA was amplified using following primers: ALP gene (forward 5′-GACCCTTGACCCCCACAAT-3′, reverse 5′-GCTCGTACTGCATGTCCCCT-3′), type-I collagen gene (forward 5′-CCCTGTCTGCTTCCTGTAAACT-3′, reverse 5′-CATGTTCGGTTGGTCAAAGATA-3′), GAPDH gene (forward 5′-GAGTCAACGGATTTGGTCGT-3′, reverse 5′-GACAAGCTTCCCGTTCTCAG-3′).

The amplification was conducted at 95° C. for 45 s, at 60° C. for 45 s, at 60° C. for 1 m and at 72° C. for 45 s. After 35 cycles, the PCR product was isolated in 1.5% agarose gel by electrophoresis at 100V for 15 m. The gel was then stained with 1 mg/ml ethidium bromide (EtBr), and visualized using Alpha Ease gel image analysis software (Alpha Innotech, San Leandro, Calif., USA).

As a result, considerable expresson of alkaline phosphtase and type-I collagen was observed in the conjugate scaffold, and this is attributable to the effect of amylopectin in the chitosan/HAp scaffold.

While the present invention has been particularly shown and described with reference to exemplary embodiments thereof, it will be understood by those skilled in the art that various changes in form and details may be made therein without departing from the spirit and scope of the invention as defined by the appended claims.

What is claimed is:

1. A porous scaffold for tissue engineering comprising chitosan, hydroxyapatite and amylopectin joined with each other in a three dimensional manner.

2. The porous scaffold for tissue engineering as set forth in claim 1, wherein the chitosan has molecular weight of 200˜350 KDa and deacetylation of 85˜95%.

3. The porous scaffold for tissue engineering as set forth in claim 1, wherein the hydroxyapatite is isolated from bones of Thunnus obesus.

4. The porous scaffold for tissue engineering as set forth in claim 1, wherein the scaffold has a porosity of 85˜95%.

5. The porous scaffold for tissue engineering as set forth in claim 1, wherein the scaffold has a pore size of 60˜500 μm.

6. The porous scaffold for tissue engineering as set forth in claim 1, wherein the scaffold comprising 50˜100 wt. % of hydroxyapatite and 50˜100 wt. % of amylopectin per 100 wt. % of chitosan.

7. A preparation method of a porous scaffold for tissue engineering, the preparation method comprising steps of:
a) dissolving chitosan in acetic acid solution;
b) adding hydroxyapatite in the chitosan solution;
c) adding amylopectin in the solution formed at step b); and
d) preparing the scaffold by freeze-drying the solution formed at step c).

8. The preparation method as set forth in claim 7, wherein the freeze-drying is conducted for 24˜48 hours.

9. The preparation method as set forth in claim 7, wherein after step a), further comprising a step of treating the chitosan solution with ultrasonic treatment for 0.5˜2 hours.

10. The preparation method as set forth in claim 7, after step d), further comprising a step of submerging the scaffold formed from the freeze-drying in a basic solution to adjust the scaffold to neutral pH.
